(19) United States
(12) Patent Application Publication
(10) Pub. No.: US 2004/0176800 A1
(43) Pub. Date: Sep. 9, 2004

(54) Barbed closure device

(76) Inventors: Joseph Francis Paraschac, San Jose, CA (US); Joshua Stafford, Menlo Park, CA (US); Tom Kelly, Fremont, CA (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/791,265

(22) Filed: Mar. 2, 2004

Related U.S. Application Data
(60) Provisional application No. 60/452,535, filed on Mar. 7, 2003.

(51) Int. Cl. A61B 17/08

(52) U.S. Cl. 606/213

(57) ABSTRACT

In accordance with the present invention there is provided devices and methods for use during post-catheterization of an arterial puncture of a blood vessel lumen such as an artery in a patient. The barbed closure device in accordance with the present invention includes an anchor member, an arm member and at least one barb member associated with the arm member, wherein the arm member is movable between a first and second position and the barb members are configured to grip tissue immediately surrounding a periphery of the artery.

### TECHNICAL FIELD

The present invention is relates generally to devices and methods for the closure of openings formed in a vessel, artery or tissue.

### BACKGROUND OF THE INVENTION

A number of diagnostic and interventional vascular procedures are now performed transluminally, where a catheter is introduced to the vascular system at a convenient access location and guided through the vascular system to a target location using established techniques. Such procedures require vascular access which is usually established using an introducer sheath according to the well known Seldinger technique, as described, for example, in William Grossman's “Cardiac Catheterization and Angiography”, 3^{rd} Ed., Lea and Febiger, Philadelphia, 1986, incorporated herein by reference.

When vascular access is no longer required, the introducer sheath must be removed and bleeding at the puncture site stopped. Once common approach for achieving hemostasis (the cessation of bleeding) is to apply external force adjacent to and upstream from the puncture site, typically by manual or “digital” compression. This approach suffers from a number of disadvantages. It is time consuming, frequently requiring one-half hour or more compression before hemostasis is assured. It is uncomfortable for the patient and frequently requires administering analgesics to be tolerable. Moreover, the application of excessive pressure can at time totally occlude the underlying blood vessel, resulting in ischemia and/or thrombosis. Following manual compression the patient is required to remain recumbent for at least six and at times as long as eighteen hours under close observation to assure continued hemostasis. During this time, renewed bleeding may occur resulting in bleeding through the tract, hematoma, and/or pseudoaneurism formation as well as arteriovenous fistula formation. These complications may require blood transfusion and/or surgical intervention.

The incidence of these complications increases when the sheath size is increased and when the patient is anticoagulated. It is clear that the standard technique for arterial closure can be risky and is expensive and onerous to the patient. While the risk of such conditions can be reduced by using highly trained individuals, such use is both expensive and inefficient.

Other approaches have been taken to address the shortcomings described above. One such device is shown and described in U.S. Pat. No. 4,744,364 which shows a device for sealing a vessel puncture in the form of a device having an expandable closure member with a filament for pulling it against the vessel wall. The closure member is held in place by a strip of tape placed on the skin to hold the filament in place. However, the closure device is still subject to movement which can cause leakage through the puncture. Additionally, if the suture becomes loose, the closure member is not retained and can flow downstream within the vessel. Further still, since the suture extends through the skin, this creates a potential pathway for infection.

Another approach to vessel closure can be seen in U.S. Pat. No. 5,545,178 wherein a resorbable collagen foam plug is deployed within the puncture tract. However, since coagulation typically takes up to twenty minutes and blood can leak in between the plug and tissue tract, manual pressure must be applied to the puncture for a period of time until the collagen plug expands within the puncture tract, thereby sealing the puncture tract.

It would therefore be advantageous to provide a device which would more quickly and effectively close openings formed in vessels, arteries and tissue. Wherein the device could be easily and readily deployed with minimal risk of leakage or becoming dislodged.

These and other objects, advantages, and features of the invention will become apparent to those persons skilled in the art upon reading the details of the methods and systems of the present invention that are more fully described below.

### SUMMARY OF THE INVENTION

The present invention provides a barbed closure device for use during post-catheterization closure of a puncture formed in a vessel wall, an arterial wall or a tissue tract. The barbed closure device includes an anchor that contacts an interior portion of an arterial wall and barbs that grip tissue immediately surrounding a periphery of the artery such as in the tissue tract above the puncture.

In accordance with the present invention there is provided a device for closing an opening formed in a vessel, the device comprising: an anchor deployable within a blood vessel lumen; at least one barb member having an undeployed position and a deployed position, wherein the barb member is moved to the deployed position when tension is applied to the device; and a body member extending between the anchor and the barb member.

In accordance with another embodiment of the present invention there is provided a device for closing an opening formed in a vessel or artery, the device comprising: an anchor portion; an arm; and at least one barb member in association with the arm.

In accordance with the present invention there is provided a method for closing an opening in an artery. The method including the steps of inserting a deployment device into an opening formed in an artery, inserting the barbed closure device of claim 1 into an artery lumen, and applying tension on a suture to deploy the barbed closure device thereby forming a fluid tight seal.

### BRIEF DESCRIPTION OF THE FIGURES

To facilitate understanding, the same reference numerals have been used (where practical) to designate similar elements that are common to the Figures. Some such numbering has, however, been omitted for the sake of drawing clarity.

FIG. 1 is a perspective view of the exemplary barbed closure device in accordance with FIG. 1, illustrating a first position;

FIG. 2 is a is a perspective view of the exemplary barbed closure device wherein the arm is moved from a first position to a second position through the application of a force;

FIG. 3 illustrates an environmental view of an exemplary embodiment of a barbed closure device as deployed within an opening in an artery or vessel;

FIG. 4 is a perspective view of the barbed closure device of FIG. 3, illustrating the deployment of the barbed closure device in accordance with the present invention;

FIG. 5 is an embodiment of the present invention illustrating a perspective view of the barbed closure device shown with reference to FIG. 4 in a fully deployed state;

FIG. 6 shows a perspective view of an alternative embodiment of a barbed closure device in accordance with the present invention;

FIG. 7 illustrates and alternative embodiment of a barbed closure device in accordance with the present invention;

FIG. 8 is an illustration of the barbed closure device of FIG. 7 in an expanded state;

FIG. 9 is a perspective view of yet another alternative embodiment of a barbed closure device in accordance with the present invention;

FIG. 10 illustrates a schematic view of the barbed closure device of FIG. 9 illustrating the deployment of the barbed closure device;

FIG. 11 illustrates a schematic view of the barbed closure device of FIG. 9 illustrating the deployment of the barbed closure device;

FIG. 12 illustrates a schematic view of the barbed closure device of FIG. 9 illustrating the deployment of the barbed closure device;

FIG. 13 illustrates a schematic view of the barbed closure device of FIG. 9 illustrating the deployment of the barbed closure device;

FIG. 14, is a perspective view of yet another embodiment of an alternative barbed closure device in accordance with the present invention; and

FIG. 15 is a perspective view of yet another embodiment of an alternative barbed closure device in accordance with the present invention.

### DETAILED DESCRIPTION OF THE PREFERRED EMBODIMENTS

Before the present invention is described in such detail, it is to be understood that this invention is not limited to particular variations set forth herein as various changes or modifications may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The present invention provides a barbed closure device for use during post-catheterization closure of an arterial puncture of a blood vessel lumen such as an artery in a patient. The barbed closure device includes an anchor member which is configured to engage an interior of an artery and at least one barb member that is configured to grip tissue or the vessel wall immediately surrounding a periphery of the artery such as in the tissue tract above the puncture on the artery. As will be discussed in greater detail with respect to the accompanying Figures, the anchor member, and the barbs are configured to close the incision or opening in the artery to form a fluid tight seal therein.

The barbed closure device in accordance with the present invention comprises an anchor member, an arm member and at least one barb member, wherein the barb member is associated with the arm member. The barbed closure device may further include an aperture disposed on the distal end of the arm member and adjacent to the barb member, wherein the aperture is configured to receive a piece of suture or a force applying member such as a wire hook or the like.

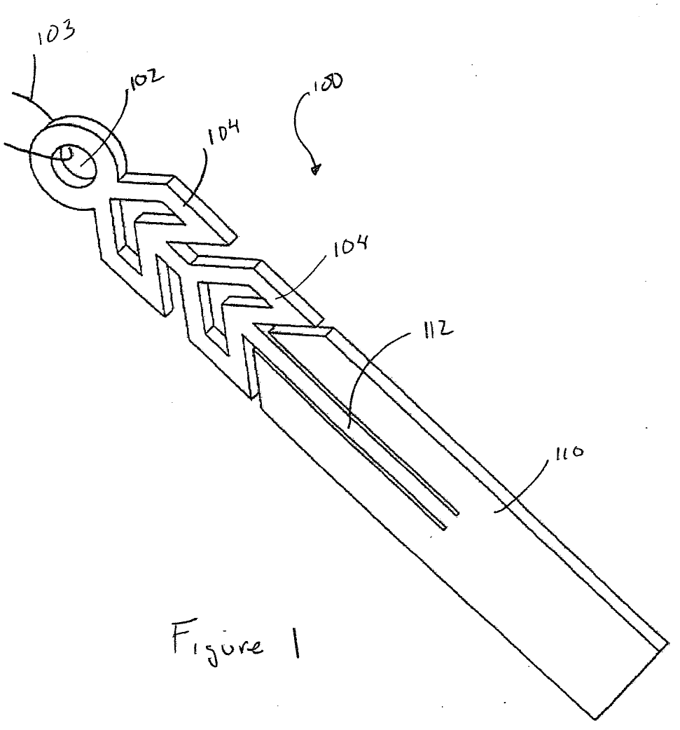

Referring now to FIG. 1, there is shown an exemplary embodiment of a barbed closure device 100 in accordance with the present invention. As shown in FIG. 1, the barbed closure device comprises an anchor member 110, an arm member 112 having a proximal end and a distal end, the proximal end in association with the anchor member, at least one barb member 104 in association with the arm member 112 and an aperture 102 disposed adjacent to the distal end of the arm member 112. As shown in FIG. 1, the barbed closure device 100 may be manufactured from sheet material, using a manufacturing process such as laser cutting, water jet cutting, stamping, machining, chemical etching, wire EDM, or similar production processes. FIG. 1 illustrates the device in an initial state after being manufactured from a sheet of material, wherein the device is generally planar. Although the barbed closure device 100 has been described as being constructed as a unitary member, it shall be understood that the device may be constructed of multiple pieces which may then be assembled to form a unitary member. For example, the arm member may be constructed separately from the anchor member and attached to the anchor member through the use of glues, welding, swaging, or similar manufacturing processes.

The barbed closure device 100 may be constructed of any biocompatible material such as series 300 stainless steel, stainless steel, Nitniol, titanium, polymers, plastics, ceramics or similar biocompatible materials. Further still, it is contemplated that the barbed closure device 100 may be constructed of a bioabsorbable material. It is further contemplated that various components of the barbed closure device 100 may be constructed of different materials. For example, the anchor member may be constructed of a bioabsorbable material, the arm member constructed of a resilient material and the barbs constructed of yet another material, or the entire barbed closure device may be constructed of a single material.

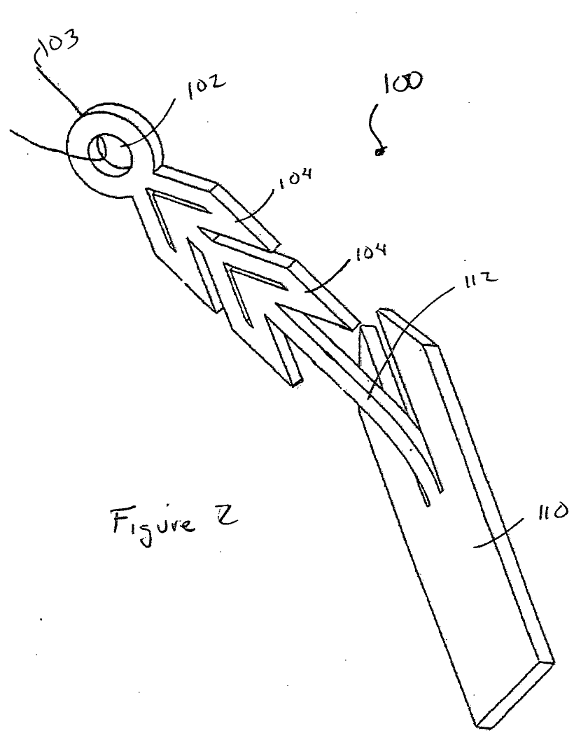

Referring now to FIG. 2, there is shown the barbed closure device 100 in accordance with one embodiment of the present invention, wherein the arm member has been moved from a first position (generally planar with the anchor member) to a second position. Wherein when disposed in the second position the arm is offset at an angle relative to the anchor member. The angle between the arm member and the anchor member may be between about 0 and 180 degrees, more preferably between about 0 and 120 degrees and most preferably between about 0 and 90 degrees. Arm 112 is movable between the first and second positions by applying a force to the distal end of the arm member while the anchor member is retained. For example, in use the anchor member 110 would be retained by the interior wall of the artery or vessel, wherein a force can then be applied to the distal end of the arm, for example by passing a suture member 103 or similar item through the aperture 102 and applying a force thereupon. The arm member moves from a first position, as shown in FIG. 1, to a second position as shown in FIG. 2 in response to the applied force. This process will be described in greater detail with reference to the methods in accordance with the present invention.

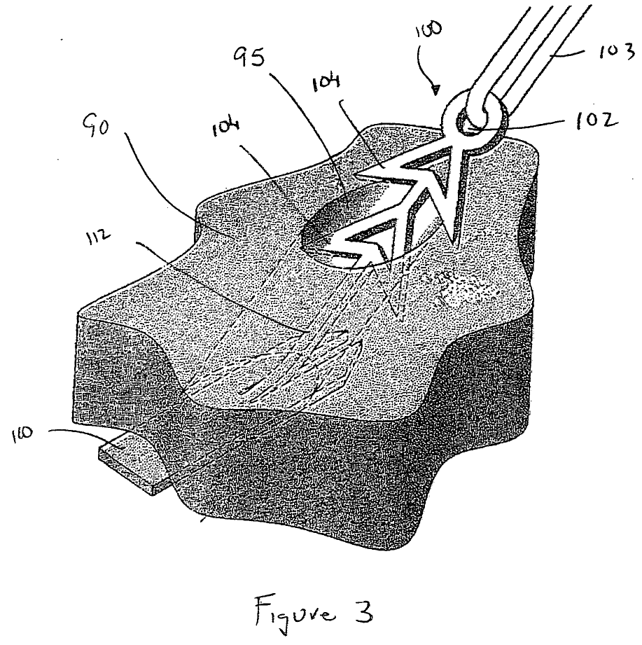

Referring now to FIG. 3, there is shown the barbed closure device 100 in an environmental view of the barbed closure device 100 being used to close a puncture 95 in a vessel 90 in accordance with the present invention. The barbed closure device 100 may be used during any procedure in which an incision or puncture is made in an artery wall such as angioplasty, an angiogram or the like. During insertion of the barbed closure device 100 into an artery, the barbed closure device 100 is loaded into a loading tube (not shown) such as a hypodermic tube or the like. The barbed closure device 100 is loaded into the loading tube in a compressed or un-expanded state such as that shown in FIG. 1, wherein the barb members 104 are tightly packed against one another. The compressed state of the barbed closure device 100 allows fitment of the barbed closure device 100 within the loading tube.

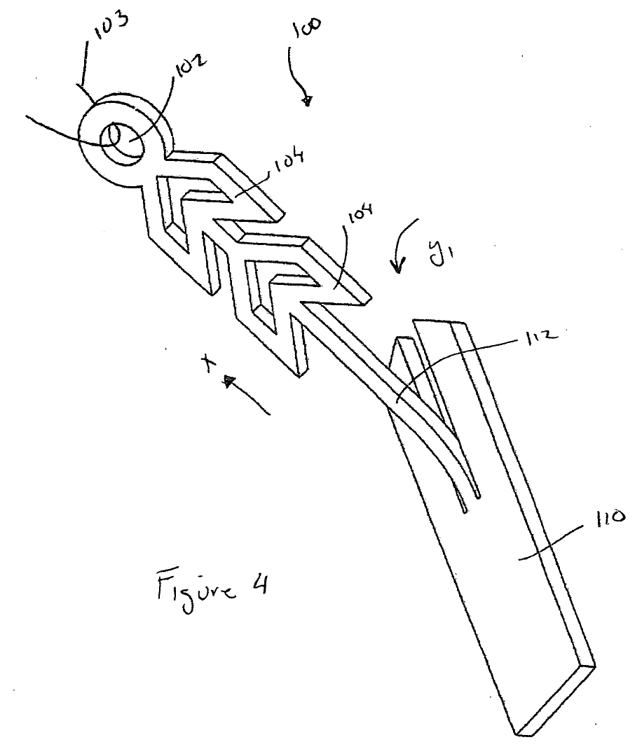

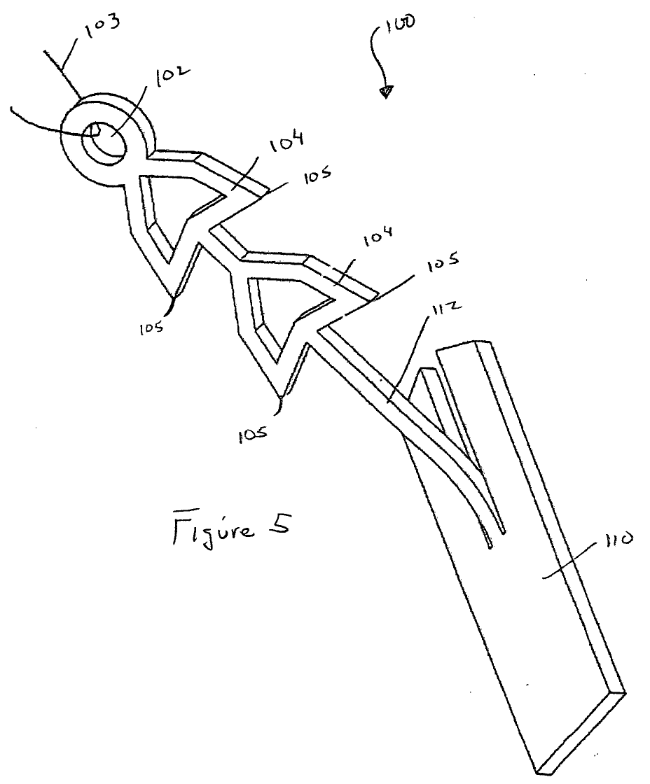

Upon insertion of the barbed closure device 100 into an artery, the loading tube is retracted while the anchor 110 and the arm 112 remain within the interior of the artery. After the loading tube is fully retracted, the arm 112 is moved from the first position to the second position by applying a force to the distal end of the arm. As described above, a force may be applied to the distal end through the use of a suture or suture material disposed through the aperture 102 or through the use of other means such as a wire hook or loop. As the arm is moved from the first position to the second position the arm may also rotate in a direction indicated by arrow Y₁ as indicated in FIG. 4. In response to the applied force, the arm member is drawn through the puncture tract formed in the artery and tissue while a top surface of the anchor member engages the inner wall 91 of the artery 90. After the arm member 112 had been deployed, a user tensions the suture 103 in the direction indicated by the directional arrow X. As a user tensions the suture 103, the barbed closure device 100 achieves the configuration as shown in FIG. 5. It is further contemplated that the loading tube may be utilized to retain the barbed closure device in a insertion position, wherein when the loading tube is removed the barbed closure device automatically moves from a first position to a second position without interaction from the user. In this example, the device would be manufactured having a “set” or pre-formed shape wherein at rest the device would be in a deployed state.

Referring now to FIG. 5, there is shown an embodiment of the present invention illustrating a perspective view of the barbed closure device 100, wherein the barbed closure device 100 is in a fully deployed condition such that the barbed closure device 100 is deformed under tension and the barb members 104 extend wider than in the undeployed configuration as shown in FIGS. 1 and 2. Upon tensioning the suture 103, the barb members 104 extend laterally or expand to be wider than in the undeployed condition. When the barb members 104 achieve this configuration, the tips 105 of the barb members grip tissue 92 immediately surrounding an outer periphery of the artery 90. It should be noted that the barb members 104 and the tips 105 may also grip subcutaneous tissue (not shown) that forms a tissue tract (not shown) above the puncture 95 shown with reference to FIG. 3. In this configuration, the barbed closure device 100 closes an incision made within an artery wherein both the anchor member 110 and the barb members 104 act to close the puncture 95 in the artery 90.

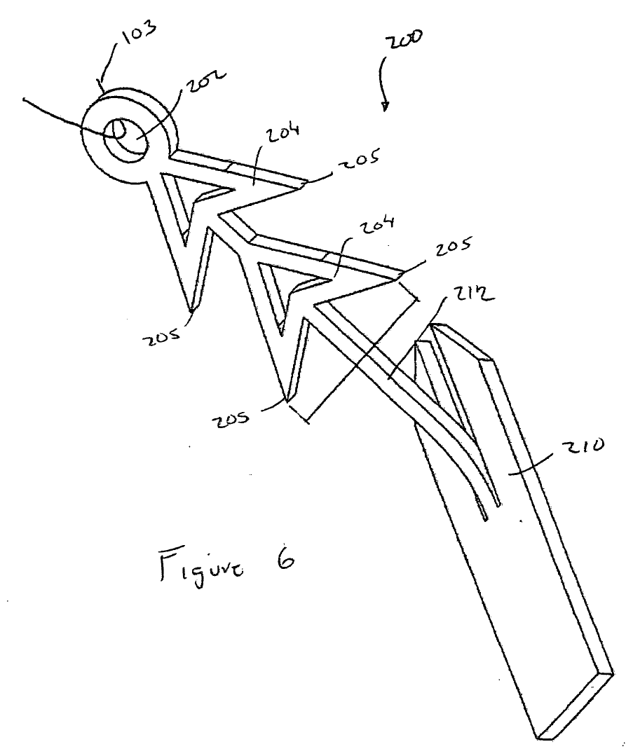

Referring now to FIG. 6, there is shown an exemplary embodiment of an alternative barbed closure device 200 in accordance with the present invention. The alternative embodiment of the barbed closure device comprises an anchor member 210, an arm member 212 having proximal and distal ends, and at least one barb member 204. As shown in FIG. 6, the barb members 204 have a shorter length such that a smaller amount of tissue is affected during closure of an artery. As such, smaller barb members provide for a smaller implant profile. Upon tensioning of the suture 103 disposed through the aperture 202, the barbed closure device 200 has a configuration shown with reference to FIG. 6.

It should be noted that the barbed closure device 200 is delivered to an artery in the same manner as previously described with respect to FIG. 3 and the barbed closure device 100. Likewise, the barbed closure device 200 is also deployed within the artery in the same manner wherein a loading tube is retracted thereby allowing engagement of the anchor member 210 with an interior of an artery wall of a patient. Moreover, the suture 103 tensions the barbed closure device 200 such that the barb members 204 achieve the configuration shown with respect to FIG. 6. In addition, the tips 205 of the barb members 204 engage with tissue immediately surrounding a periphery of an artery upon tensioning of the suture 103.

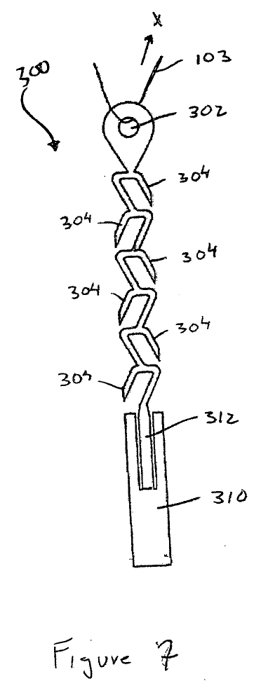

Referring now to FIG. 7, there is shown a schematic of a barbed closure device 300 in accordance with yet another alternative embodiment of a barbed closure in accordance with the present invention. In this embodiment, the barbed closure device 300 includes an anchor member 310, an arm member 312 having a proximal and distal end, and at least one barb member 304 associated with the arm member. The barbed closure device 300 further includes an aperture 302 disposed adjacent to the distal end of the arm member 312. As shown in FIG. 7, the barbed closure device 300 is shown in a compressed state similar to that previously described with regard to the barbed closure device 100, such that the barbed closure device 300 may be placed in a loading tube for delivery. Upon delivery of the barbed closure device 300 into an artery of the patient, the loading tube is retracted and the anchor member 310 rotates into position to engage with an interior wall of the artery as shown in FIG. 7 and as previously discussed. Moreover, once the anchor member 310 engaged with an interior of the artery of the patient, a user tensions the suture 103 in a direction indicated by directional X of FIG. 7, thereby achieving the configuration as shown with respect to FIG. 8. As may be seen with respect to FIG. 8, the barb members 304 are in a deployed state wherein a barb tip 305 engages with tissue adjacent an outer periphery of the artery in which the anchor member 310 engages.

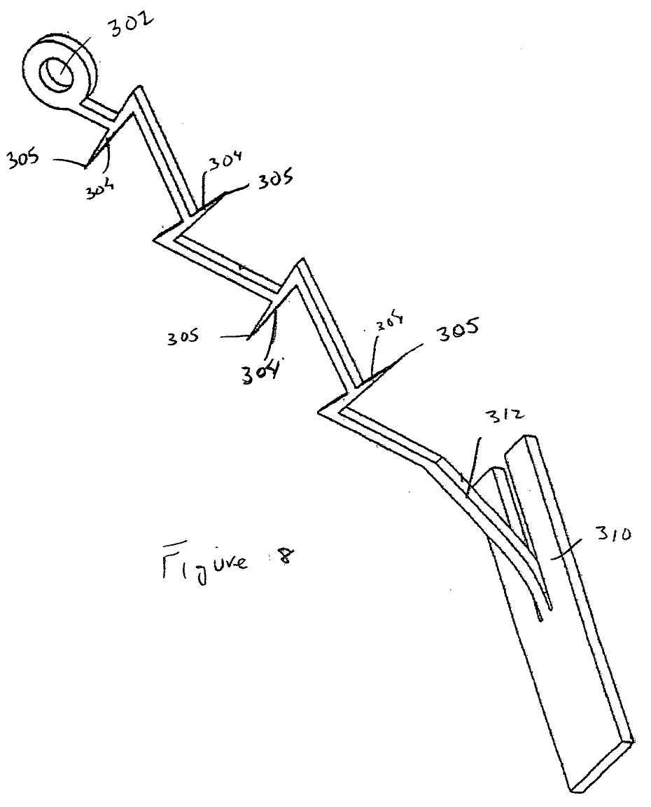

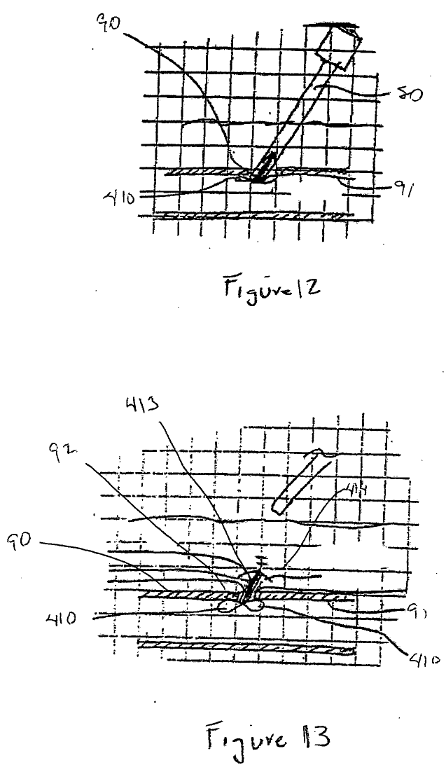

Referring now to FIG. 9 there is shown a schematic view of a barbed closure device 400 in accordance with an alternative embodiment of the present invention. The barbed closure device 400 includes barb members 405, a plug member 415 and an anchor member 410. The barbed closure device 400 may be inserted into an artery of a patient with an introducer sheath, as shown in FIG. 12.

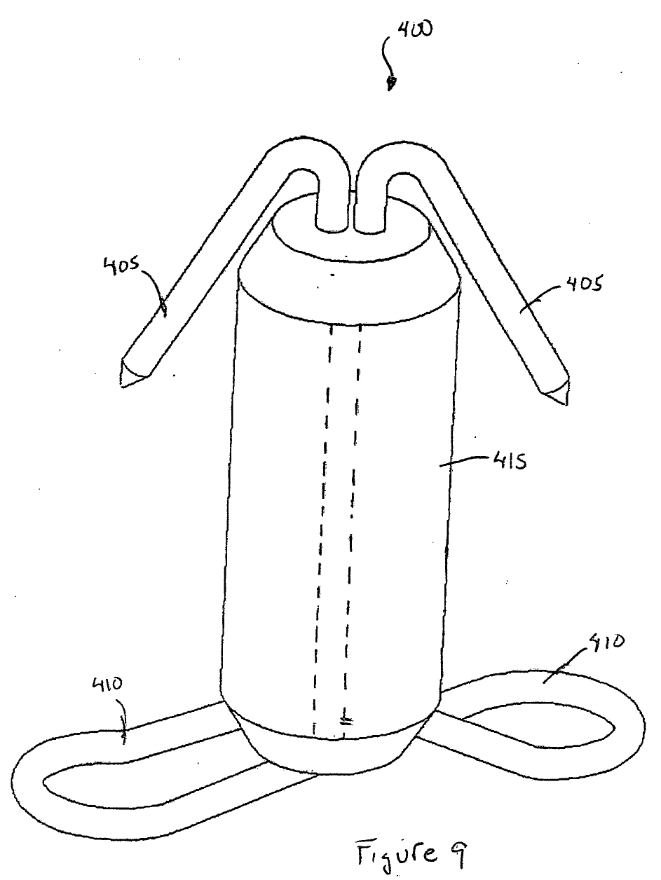

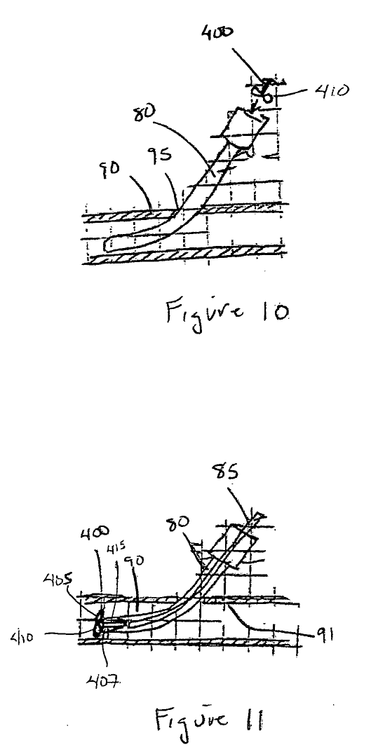

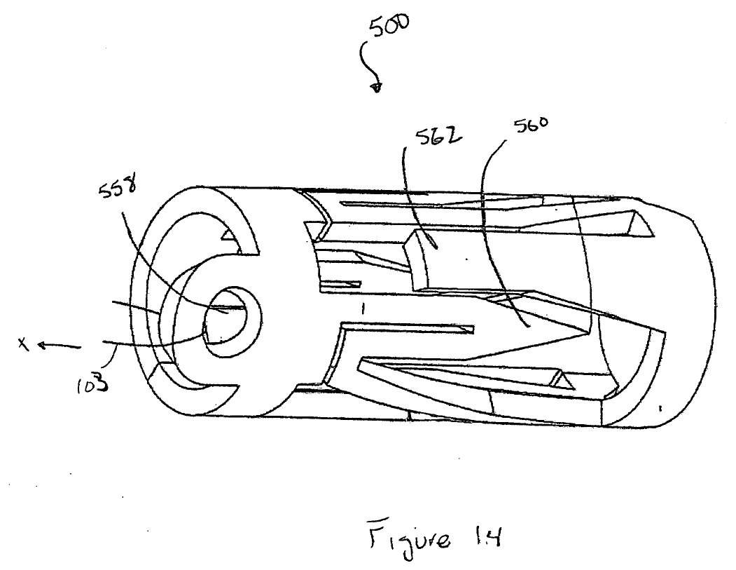

Referring now to FIG. 10, there is shown a schematic view of an alternative method of delivering the barbed closure device 400 to an artery 90 through an incision 95. In this embodiment, the barbed closure device 400 is delivered to the artery 90 with an introducer sheath 80. As may be seen with reference to FIG. 11, a push rod 85 pushes the barbed closure device 400 through the introducer sheath 80 and into the artery 90. Once the barbed closure device 400 enters into the artery 90, a user retracts the introducer sheath 80. Upon retraction of the introducer sheath 80, an anchor 410 of the barbed closure device 400 expands and engages with an inside surface 91 of the artery 90 as shown with reference to FIG. 12. The anchor 410 is connected to the barb member 405 via a shaft 407 disposed within the plug 415 wherein the plug 415 is around the shaft. As a user continues to retract the introducer sheath 80, the barb member 405 of the barbed closure device 400 are exposed and expand from the barbed closure device 400, which may be seen with reference to FIG. 13. The barbed closure device 400 may also include the plug 410 which is configured to fit into the incision 95 of the artery 90. The barb members 405 grip subcutaneous tissue 414 of a patient on a first side of the plug 415 and the anchor 410 engages with the surface 92 of the artery 90, thereby securing the plug 415 within the incision 95 of the artery 90.

In the embodiment shown with reference to FIGS. 9-13, the barb members 405 and the anchor 410 may be constructed from any biocompatible material such as those previously described. The plug 415 may be a plastic tube which allows for passage of a wire in order to control the deployment of both barb members 405 and the anchor 410. The plug 415 may include a tether which ensures hemostasis prior to release and allows a snug fit of both the barb members 405 and the anchor 410. Exemplary plug materials may include collagen, polyglycolic acid, polylactic acid, or any suitable bioabsorbable material. Other suitable materials may be biocompatible materials such as polyvinyl alcohol or nylon or the like.

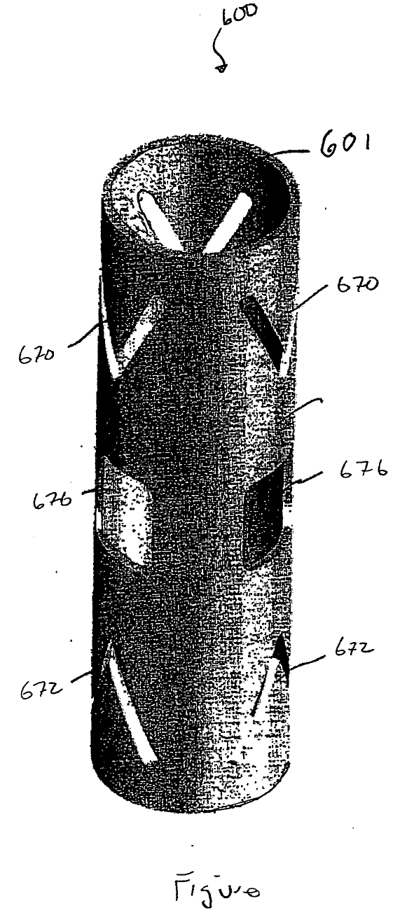

Referring now to FIG. 14, there is shown a perspective view of a barbed closure device 500 in accordance with an alternative embodiment of the present invention. The barbed closure device 500 includes an aperture 558, an anchor member 562 and a radial barb 560. During delivery of the barbed closure device 500, a loading tube, in which the barbed closure device 500 resides during delivery is retracted and the anchor 562 folds down. The anchor 562 folds down and engages with an inner wall of an artery requiring closure of a patient similar to the anchor members previously discussed with regard to the other embodiments of the present invention. Is should be noted that in one embodiment of the invention shown with reference to FIG. 14, the barbed closure device 500 may include more than one anchor 562 which engages the inner wall of an artery. After deployment of the anchor 562, the suture 103 is tensioned in a direction indicated by the directional arrow X, thereby deploying the radial barbs 560. As discussed with reference to FIGS. 1 and 2 the barb members 104, the radial barbs 560 grip tissue immediately surrounding a periphery of an artery requiring closure. More specifically, the radial barbs 560 include a tip 561 that grips tissue surrounding the artery.

Referring now to FIG. 15, there is shown a perspective view of a barbed closure device 600 in accordance with an alternative embodiment of the present invention. The barbed closure device 600 includes proximal barb members 670, distal barb members 672, and cutouts 676. During delivery of the barbed closure device 600, the barbed closure device 600 is positioned within a loading tube (not shown) and includes a configuration shown with respect to FIG. 15. Upon retraction of the loading tube and after insertion of the barbed closure device 600 within an artery requiring closure, the distal barb members 672 deploy in a similar manner to the anchor member of the alternative embodiments. After deployment, the distal barbs 672 engage with an interior surface of the artery. It should be noted that the distal barbs 672 prevent proximal migration of the barbed closure device 600 subsequent to deployment of the barbed closure device 600 within the patient. In addition, after the distal barbs 672 deploy, the proximal barbs 670 are also deployed once the loading tube is retracted from the proximal barbs 670. The proximal barbs 670 grip subcutaneous tissue proximal the artery wall of the artery requiring closure. The proximal barbs 670 prevent distal migration of the barbed closure device 600 and counterbalance forces applied to the barbed closure device 600 by the distal barbs 672.

The barbed closure device 600 also includes cutouts 676. In one embodiment, the cutouts 676 are hollow portions within the barbed closure device 600 which may be used to insert collagen into a middle portion 601 of the barbed closure device 600. In addition, an insert molded plastic plug may also be inserted through the cutout 676 into the middle portion 601 of the barbed closure device 600 such that the middle portion 601 prevents migration of fluid through the barbed closure device 600 upon closure of an inclusion within the artery. In addition, a solid plug within the middle portion 601 may be formed during formation of the cutout 676 where material removed during formation of the cutout 676 is folded inwardly towards the middle portion 601 thereby creating a solid plug within the barbed closure device 600.

It is further contemplated with regard to the barbed closure devices 400 and 500 of FIGS. 14, 15 that a special delivery device may be utilized to deploy the barbed members of each of these embodiments in addition to providing a fluid tight seal therein. For example, the delivery device may include a shaft member which extends through a central aperture of each of the devices and including an expandable member or a wedge member such that the expandable member or wedge member engages the barb members of each device respectively wherein upon actuation of the expandable member or wedge member thereby deploys the barb members and seals the central aperture providing a fluid tight seal therein.

It shall be understood that in accordance with the embodiments of the present invention that the barbed closure devices described herein may be formed through molding, casting, blanking, laser cutting, chemical etching, stamping, milling or other similar processes. Further still, although the barbed closure devices have been described herein requiring an external force to engage the anchor members with the artery wall or the barb members with the tissue it is contemplated that the devices may be manufactured having a resilience or a spring force contained therein, such that the barb and/or anchor members may deploy upon insertion into the puncture or artery.

The instant invention is shown and described herein in what is considered to be the most practical, and preferred embodiments. It is recognized, however, that departures may be made there from, which are within the scope of the invention, and that obvious modifications will occur to one skilled in the art upon reading this disclosure.

What is claimed is:

1. A device for closing an opening formed in a vessel, the device comprising:
an anchor deployable within a blood vessel lumen;
at least one barb member having an undeployed position and a deployed position, wherein the barb member is moved to the deployed position when tension is applied to the device; and
a body member extending between the anchor and the barb member.

2. The device of claim 1, the device further comprising:
a plug disposed on the body member.

3. The device of claim 1, wherein in the undeployed position the device is generally planar.

4. The device of claim 1, wherein the device further includes an aperture formed adjacent to the barb member.

5. The device of claim 4, the device further including a suture disposed through said aperture.

6. The device of claim 1, wherein the device is constructed of a bioabsorbable material.

7. The device of claim 1, wherein the device is constructed of a material chosen from the group consisting of: stainless steel, titanium, nitinol, plastic and ceramic.

8. The device of claim 1, wherein the device is manufactured from sheet material.

9. The device of claim 1, further including a biocompatible coating disposed thereon.

10. A device for closing an opening formed in a vessel or artery, the device comprising:
an anchor portion;
an arm; and
at least one barb member in association with said arm.

11. The device of claim 10, wherein the arm is movable between a first and second position, wherein in said first position of said arm is generally planar with said anchor portion.

12. The device of claim 11, wherein said second position of said arm forms an angle between said anchor portion and said arm.

13. The device of claim 12, wherein said arm is movable between said first and second position through the application of a force to a distal end of said arm.

14. The device of claim 13, wherein said barb member moves from a first position to a second position in response to said force.

15. The device of claim 14, further including a sealing member in association with said anchor member.

16. The device of claim 1, wherein the barb member in the deployed position has a greater width than in the first position.

17. The device of claim 14, wherein said barb member becomes wider between said first and second positions.

18. A barbed closure device, the barbed closure device comprising:
a main body having a generally cylindrical shape; and
at least one barbed member, wherein said barb member is radially movable between a first and second position.

19. The barbed closure device of claim 18, further including a plug disposed within an aperture formed axially within the main body.

20. The barbed closure device of claim 19, wherein said plug is movable between a first and second position and configured to move said barb member between said first and second position.
